(19) United States
(12) Patent Application Publication
(10) Pub. No.: US 2023/0017101 A1
(43) Pub. Date: Jan. 19, 2023

(54) Molecule Sensor Component and Method for Manufacturing Same

(71) Applicant: Northeastern University, Boston, MA (US)

(72) Inventors: Mohammadamin Alibakhshi, Boston, MA (US); Meni Wanunu, Needham, MA (US); Xinqi Kang, Boston, MA (US)

(21) Appl. No.: 17/779,995

(22) PCT Filed: Nov. 27, 2020
(86) PCT No.: PCT/US2020/062520

§ 371 Date: May 25, 2022

Related U.S. Application Data
(60) Provisional application No. 62/941,582, filed on Nov. 27, 2019.

(51) Int. Cl. B01D 67/00

(52) U.S. Cl. CPC B01D 67/0072; B01D 67/0053; B01D 2323/08; B01D 2323/35; B01D 2325/02

(57) ABSTRACT

A method for manufacturing a component and a component are provided for sensing a molecule. The method includes controlling a temperature during a reaction of two gases that react to produce a crystalline film spanning at least a cross-sectional area of a nanoaperture defined by a substrate among an array of nanoapertures aligned with crater structures defined by the substrate. A unique chemical vapor deposition (CVD) method that introduces a first gas and a second gas allows for formation of the crystalline film. When used in a molecule sensor, the component enables a user to record double-stranded DNA (dsDNA) translocations at unprecedented high (e.g., 1 MHz) bandwidths. The method for manufacturing the component enables development of applications requiring single-layer membranes built at- scale and enables high throughput 2-dimensional (2D) nanofluidics and nanopore studies.

### RELATED APPLICATION(S)

This application claims the benefit of U.S. Provisional Application No. 62/941,582, filed on Nov. 27, 2019 the entire teachings of which are incorporated herein by reference.

### BACKGROUND

Two-dimensional (2D) materials, owing to their extremely low thickness, are ideal materials for nanopores with optimal detection sensitivity and resolution. Among 2D materials, molybdenum disulfide (MoS₂) has gained significant traction as a more suitable nanopore material compared to graphene, which is much more hydrophobic. Performing experiments using 2D nanopores, however, remains challenging due to the lack of methods for scaled-up fabrication of high-quality freestanding membranes.

### SUMMARY

Presented herein is a site-directed, scaled-up synthesis of MoS₂ freestanding membranes on nanoapertures on substrates (e.g., 2- to 12-inch substrate substrates) with 75% yields. A unique chemical vapor deposition (CVD) method that introduces sulfur and molybdenum dioxide vapors from both sides of sub-100 nm nanoapertures allows for the exclusive formation of freestanding membranes across the nanoapertures. This results in nucleation and growth near the nanoaperture edges and into the nanoapertures, followed by nanoaperture decoration with MoS₂, which proceeds until a critical flake radius of curvature is achieved, after which fully-spanning freestanding membranes form (i.e., a cross-sectional area of the nanoaperture is filled with a crystalline film extending from a nucleus at the nanoaperture edges).

Intentionally blocking flow of reagents through the nanoapertures eliminates a high nucleation density around the nanoapertures, thus guaranteeing highly-crystalline monolayer MoS₂ membranes. The in-situ grown membranes along with facile membrane wetting and nanopore formation using dielectric breakdown enabled an embodiment of the invention to record dsDNA translocations at unprecedented high 1 MHz bandwidths. The methods presented herein are useful toward development of many applications requiring single-layer membranes built at-scale and enable high-throughput 2D nanofluidics and nanopores studies.

Presented herein is a new approach that, while scalable, does not require sophisticated surface preparations and guarantees growth requirements on the nanoaperture. Example embodiments demonstrate this method in a scaled-up manner. Embodiments of the invention enable new nanopore-based DNA sequencing techniques that can perform long DNA reads at much faster rates compared to competing technologies by pulling a DNA strand through a nanometer-size pore. A small size of the final device (pocket-size), as well as the fast response of embodiments disclosed herein, is very appealing for various applications. Moreover, embodiments are less costly compared to existing techniques as this method does not require expensive labeling and reagents. Finally, the shelf life of such pores can be much greater than organic-based pore/membrane systems. For example, in existing MinION, shelf life of nanopores is only approximately 10 weeks.

In an embodiment, a method of manufacturing a component for a molecule sensor comprises exposing a substrate to a first gas and a second gas and controlling the temperature of the first gas and the second gas. The substrate defines an array of crater structures and nanoapertures aligned therewith, the gases being at a temperature that induces a reaction that produces a nucleus coupled to a surface of the substrate at the nanoapertures and forms a curvature into the nanoapertures. Controlling the temperature of the first and the second gas continues the reaction at least until a formation of a crystalline film of a solid product of the gases extends from the nucleus and fills a cross-sectional area of at least a subset of the nanoapertures.

In some embodiments, controlling the temperature is performed as a function of a diameter of the nanoaperture. In some embodiments, controlling the temperature is performed as a function of a ratio between the diameter of the nanoaperture and a layer of thickness of the crystalline film. In some embodiments, controlling the temperature is performed as a function of a geometry of the cross-sectional area of the nanoaperture, the geometry selected from a group consisting of circular, ovular, rectangular, polygonal, curvilinear, or a combination thereof.

In some embodiments, the first gas includes a sulfur vapor and the second gas includes a molybdenum dioxide vapor. In such embodiments, the nucleus includes molybdenum disulfide. In some embodiments, the nucleus is coupled to the substrate via a membrane.

In some embodiments, the substrate is a target substrate, and the method of manufacturing may further include positioning a backing substrate in parallel arrangement with the target substrate, wherein the arrangement of the target substrate and the backing substrate define a gap in which the first gas flows at a controllable rate into at least a subset of the craters of the array of crater structures. In these embodiments, the gap has a dimension that controls a flow rate of the first gas into at least the subset of craters sufficient for the first gas to react with the second gas to produce the crystalline film at at least the subset of the nanoapertures.

In some embodiments, the substrate is a backing substrate, and further comprises positioning a source substrate in parallel arrangement with the backing substrate. The arrangement of the backing substrate and the source substrate define a gap in which the first gas flows at a controllable rate. The gap is configured to retain the first gas for a time sufficient for the first gas to react with the second gas to produce the crystalline film at at least the subset of the nanoapertures. The embodiments may further include a source substrate, and the method of manufacturing may further comprise (i) coating the source substrate with a chemical agent that produces the second gas at a given temperature and (ii) aligning the source substrate in offset parallel arrangement of the target substrate in a plane opposite the target substrate relative to a plane of the backing substrate.

Embodiments may further include removing a sacrificial layer that is coupled to the substrate at a location between a given crater structure and a corresponding crystalline film.

Some embodiments further comprise pre-forming the substrate by pre-applying a pattern of positive resist on the substrate and exposing the substrate to an electron beam to form the array of the crater structures in the substrate. Some embodiments further comprise exposing the substrate to a solution containing between about 5% to about 10% hydrogen for up to ten hours at pressure ranging from 50 Torr to 100 Torr at a temperature sufficient to stabilize crystals in the cross-sectional area of the nanoapertures.

In some embodiments, exposing the substrate to the first gas and the second gas is performed for a length of time known to induce controlled growth of the crystalline film at the nanoapertures. In some embodiments, the controlled growth of the crystalline film starts at a distance offset from a given crater, allowing the first gas to enter the given crater through a gap defined by the substrate. Some embodiments further comprise applying an electric field to the crystalline film at a level that produces a nanopore therethrough.

Some embodiments further comprise separating the array of crater structures into individual components, wherein each component include a respective portion of the substrate, a respective crater, and crystalline film. Some embodiments further comprise packaging an individual component into a housing that forms a molecule sensor.

In an embodiment, a component for a molecule sensor comprises a substrate, a nucleus, and a crystalline film. The substrate defines an array of crater structures and nanoapertures aligned therewith. The nucleus is coupled to the substrate at the nanoapertures and forms a curvature into the nanoapertures. The crystalline film extends from the nucleus and fills a cross-sectional area of at least a subset of the nanoapertures.

In some embodiments, the crystalline film defines a respective nanopore through which a molecule may pass. In some embodiments, the nanopore has a diameter from about 50 nm to about 200 nm. In some embodiments, the curvature is defined by layers of the nucleus at the nanoaperture.

The nucleus may be a product of a sulfur vapor and a molybdenum dioxide vapor. The nucleus may be coupled to the substrate via a membrane.

Another example embodiment of the invention is a molecule sensor that includes a substrate, a nucleus, and a crystalline film. In this example embodiment, the substrate defines a crater structure and nanoaperture aligned therewith. The nucleus is coupled to the substrate and forms a curvature into the nanoaperture. The crystalline film extends from the nucleus and fills a cross-sectional area of the nanoaperture. The crystalline film also defines a nanopore with a dimension sufficient to enable a molecule to pass therethrough. The crystalline film may be at least partially below a surface of the substrate within the nanoaperture.

The molecule sensor may also include electrodes that, when energized, cause the molecule to pass through the nanopore and a sensor that is configured to detect a change of an electrical signal that indicates that the molecule entered, is within, or passed through the nanopore.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A depicts a substrate with individual components, in accordance with an embodiment of the invention.

FIG. 1B is a schematic of an arrangement of a backing substrate and a source substrate defining a gap and the gases therein giving rise to growth of a crystalline film, in accordance with an embodiment of the invention.

FIG. 1C is a schematic of the backing substrate, in accordance with an embodiment of the invention.

FIG. 1D depicts a scanning electron micrograph image of a freestanding SiN membrane after selective crystalline film growth, a nanoaperture covered with a monolayer of crystalline film, and a close view of crystalline film layers at the nanoaperture circumference, in accordance with an embodiment of the invention.

FIG. 1E is a faceted nanoaperture edge formed by folding of an MoS₂ layer into the nanoaperture, in accordance with an embodiment of the invention.

FIG. 1F depicts a schematic of crystalline film folding at the nanoaperture edge, in accordance with an embodiment of the invention.

FIG. 1G is an image of a crystalline film growth in the immediate vicinity of nanoapertures indicating the edge crystalline film folds back onto the substrate, in accordance with an embodiment of the invention.

FIG. 1H depicts a close view of the nanoaperture edge, in accordance with an embodiment of the invention.

FIG. 1I depicts a crystalline film after drilling nanopores using a STEM probe, in accordance with an embodiment of the invention.

FIG. 2A depicts a sulfur boat, a source substrate and a backing substrate in a tube CVD furnace, in accordance with an embodiment of the invention.

FIG. 2B-C depicts simulated temperature fields inside the tube for two different furnace configurations, in accordance with an embodiment of the invention.

FIG. 2D depicts temperature profiles along the tube at two locations for the configurations, in accordance with an embodiment of the invention.

FIG. 2E depicts atomic force microscopy of a crystalline film, in accordance with an embodiment of the invention.

FIG. 2F depicts a photoluminescence emission spectrum of a crystalline film on a substrate, in accordance with an embodiment of the invention.

FIG. 2G depicts a photoluminescence emission spectrum of a monolayer and bilayer of crystalline film of grown on a substrate, in accordance with an embodiment of the invention.

FIG. 2H depicts a Raman spectrum of a monolayer and bilayer crystalline film grown on a substrate, in accordance with an embodiment of the invention.

FIG. 3A depicts a schematic of crystalline film growth on a substrate, in accordance with an embodiment of the invention.

FIG. 3B depicts a scanning electron microscope image of a crystalline film grown on a substrate patterned with microcavities, in accordance with an embodiment of the invention.

FIG. 3C depicts a crystalline film grown on a substrate, showing the manner in which the crystalline film coats the substrate, in accordance with an embodiment of the invention.

FIG. 3D depicts a uniform coating of a source substrate with a solution, in accordance with an embodiment of the invention.

FIG. 3E depicts a container devised for horizontal positioning of the substrates in a tube and controlling the gap between the two substrates, in accordance with an embodiment of the invention.

FIG. 3F-G depicts a map of crystalline film coverage on a representative growth which improves uniformity by allowing the first gas to diffuse through the substrate, in accordance with an embodiment of the invention.

FIG. 3H depicts a schematic of a nonselective growth method, in accordance with an embodiment of the invention.

FIG. 3I depicts a scanning electron microscope image of a crystalline film grown on a membrane, covering the an nanoaperture, in accordance with an embodiment of the invention.

FIG. 3J depicts a freestanding membrane fabricated with the nonselective growth method, in accordance with an embodiment of the invention.

FIG. 3K-L depict images of water receding from a crystalline film-containing substrate before and after annealing, illustrating the transition from a hydrophilic to semi-hydrophobic crystalline film grown on a substrate, in accordance with an embodiment of the invention.

FIG. 4A depicts current versus voltage measurements for different nanopores with diameters in the range of two to four nanometers, in accordance with an embodiment of the invention.

FIG. 4B depicts current traces recorded at 0 mV and 200 mV, and lowpass filtered at different frequencies, in accordance with an embodiment of the invention.

FIG. 4C depicts noise spectra of the ionic current recorded at different applied voltages, in accordance with an embodiment of the invention.

FIG. 4D depicts a current trace of 4 nm diameter nanopore at 300 mV in the presence of 500 base-pair double-stranded DNA, recorded at 1 MHz bandwidth, in accordance with an embodiment of the invention.

FIG. 4E depicts representative translocation events occurring at different timescales, in accordance with an embodiment of the invention.

FIG. 4F depicts a scatter plot of fractional current blockade versus dwell time indicating two different populations corresponding to translocation events and DNA collision with the nanopore, in accordance with an embodiment of the invention.

FIG. 4G depicts distributions of fractional current blockades, in accordance with an embodiment of the invention.

FIG. 4H depicts dwell times for the data presented in FIG. 4F, which shows the characteristic timescales and signal amplitudes of collisions and translocations, in accordance with an embodiment of the invention.

FIG. 5 depicts a CVD setup used for growth showing the position of the furnaces and reagents, in accordance with an embodiment of the invention.

FIG. 6A-B depicts axial velocity fields for two furnace configurations, wherein the large temperature gradients give rise to strong rotational flows and leads to significant heat loss from the exposed areas, in accordance with an embodiment of the invention.

FIG. 6C depicts the temperature profile used for CVD crystalline film growth, in accordance with an embodiment of the invention.

FIG. 7A depicts an optical microscope image of crystals, in accordance with an embodiment of the invention.

FIG. 7B depicts a PL image of crystals, in accordance with an embodiment of the invention.

FIG. 8A depicts a faceted nanoaperture edge formed by folding of a mono/bilayer crystalline film into a circular nanoaperture, in accordance with an embodiment of the invention.

FIG. 8B depicts crystalline film growth after treatment of the surface with a buffered oxide etch, in accordance with an embodiment of the invention.

FIG. 9 depicts an area with almost complete crystalline film coverage wherein crystals grown and merge to form the crystalline film, in accordance with an embodiment of the invention.

FIG. 10 depicts a freestanding crystalline film formed using the nonselective method, in accordance with an embodiment of the invention.

FIG. 11A depicts a crystalline film formed on a nanoaperture, in accordance with an embodiment of the invention.

FIG. 11B depicts lithography steps for implementing a buffering oxide etch test of the crystalline film, in accordance with an embodiment of the invention.

FIG. 11C depicts a scanning electron microscope image after the buffer oxide etch, in accordance with an embodiment of the invention.

FIG. 12A depicts a large freestanding crystalline film getting pulled during the drying process after etching the solution on the source substrate, in accordance with an embodiment of the invention.

FIG. 12B-C depicts two instances of crystals being completely pulled through the nanoapertures during the drying process, which was not observed in 100 nm nanoapertures, in accordance with an embodiment of the invention.

FIG. 13A depicts current trace of a nanopore recorded at 100 mV, and lowpass filtered at different cutoff frequencies, in accordance with an embodiment of the invention.

FIG. 13B depicts a noise spectrum of the ionic current from the same nanopore recorded at different applied voltages, in accordance with an embodiment of the invention.

FIG. 14 depicts a calculated fractional current blockade versus diameter of a crystalline film monolayer nanopore when a dsDNA translocates through the nanopore, in accordance with an embodiment of the invention.

FIG. 15 depicts a polarized light (PL) microscopy image from an area on a substrate, in accordance with an embodiment of the invention.

### DETAILED DESCRIPTION

A description of example embodiments follows.

The teachings of all patents, published applications and references cited herein are incorporated by reference in their entirety.

Applying protein nanopores as single-molecule third-generation sequencers has demonstrated great promise for fast and low-cost DNA/RNA sequencing, spurring the search for alternative pore materials that exhibit greater mechanical robustness, sharper pore geometries for improved resolution, and the ability to achieve higher throughput by massively-parallel fabrication methods. Solid-state nanopores with thicknesses that are comparable to the size of single nucleotides can revolutionize sequencing by enabling readout of shorter k-mers than current protein-based nanopores. However, solid-state nanopores, which have been dominated by silicon nitride (SiN), have proven to be less stable over time than protein nanopores because of limited chemical stability in electrolyte solution, particularly at membrane thicknesses that approach 1-5 nm. This limited stability of high-resolution SiN-based nanopores (and other ceramic-based pores) set the stage for exploring various two-dimensional (2D) materials as possible replacements. Due to their crystalline atomically-thin nature, the family of 2D materials which includes graphene, hexagonal boron nitride, and transition metal dichalcogenides (TMDs) such as molybdenum disulfide (MoS₂) and tungsten disulfide, exhibit immense mechanical strength as well as exotic electronic and optical properties. Investigation of graphene nanopores for single-molecule sensing has demonstrated that their hydrophobicity leads to pore instabilities and large values of low-frequency noise (1/f) during electrical measurements. In contrast, pores in TMD membranes such as MoS₂ are less hydrophobic and exhibit lower noise values than graphene pores, at the cost of a moderate increase in pore thickness. The thickness of monolayer MoS₂ nanopores accommodates 1-2 DNA/RNA nucleotides at a time (assuming a stretched single strand in the pore), and thus, higher resolution than state-of-the-art protein pores could be achieved by direct trans-pore ion current measurements, or by coupling DNA translocation through the pore to transverse electronic current measurements, where conductance modulations in the MoS₂ layer during DNA transport are used for base calling.

While several studies employing MoS₂ nanopores have been reported to date, such as detecting DNA and its topological variations, differentiating DNA monomers/homopolymers, and detecting DNA methylation, the overall number of devices made and datasets reported are relatively limited as compared to SiN-based or biological pores, owing to the major obstacle of producing freestanding MoS₂ membranes at high throughput. Typically, MoS₂ nanopore studies are carried out by manually transferring MoS₂ flakes onto nanoapertures, which complicates device manufacture process and introduces polymer contaminations onto the membranes.

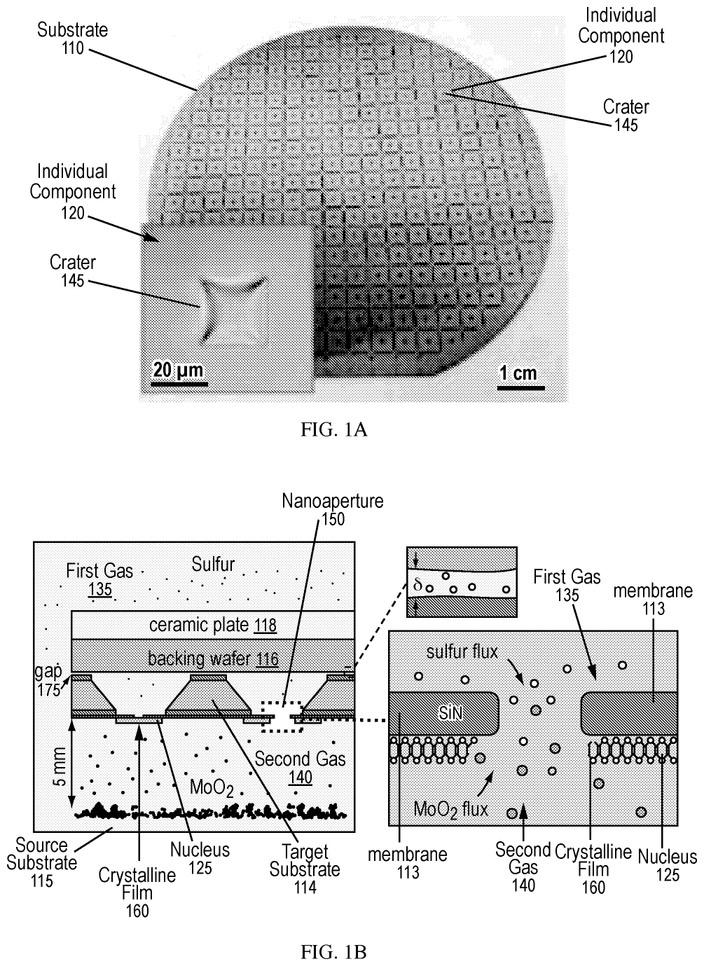

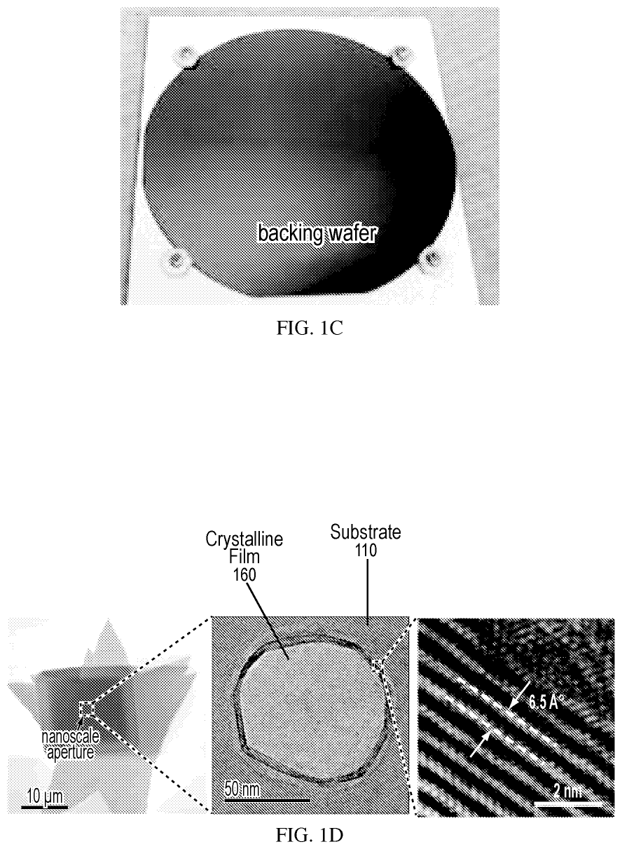

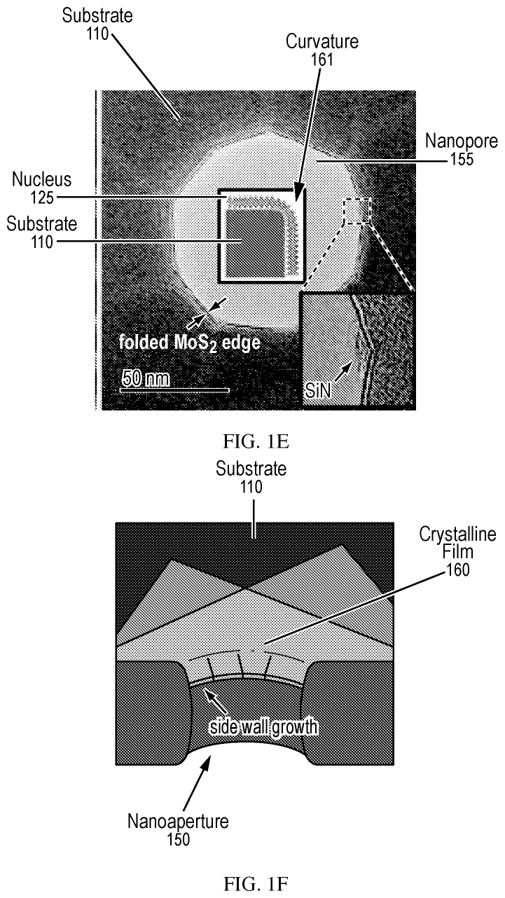

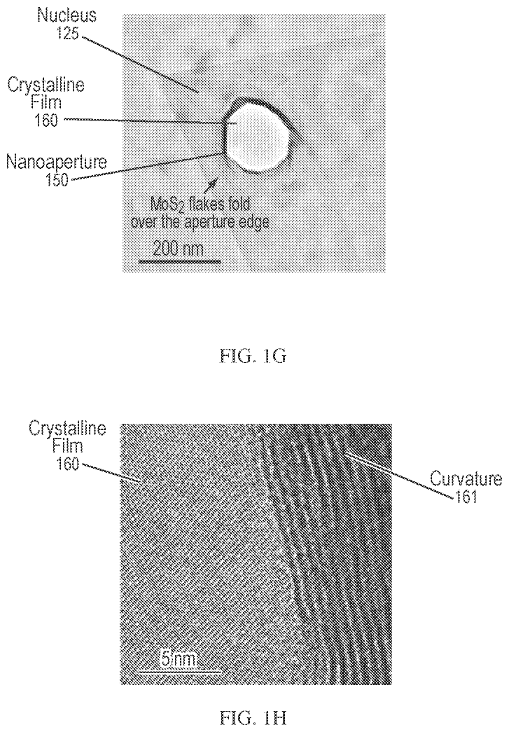

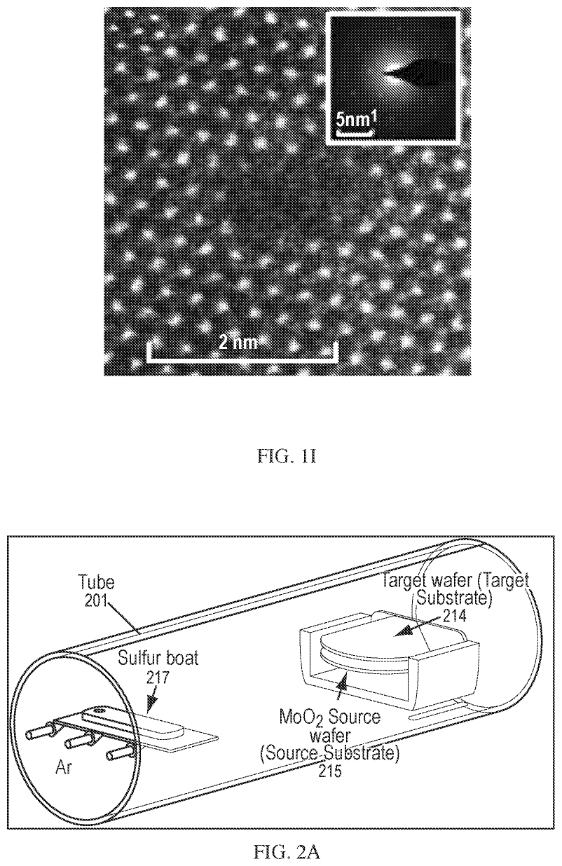

In an embodiment, and as illustrated in FIGS. 1A-B, a method of manufacturing a component for a molecule sensor comprises exposing a substrate 110 to a first gas 135 and a second gas 140 and controlling the temperature of the first gas 135 and the second gas 140. The substrate 110 defines an array of crater 145 structures and nanoapertures 150 aligned therewith, the gases being at a temperature that induces a reaction that produces a nucleus 125 coupled to a surface of the substrate 110 at the nanoapertures 150 and forms a curvature 161, as seen in FIG. 1E, into the nanoapertures 150.

Extending from an end of the nucleus 125 is a crystalline film 160. As described below in reference to later diagrams, the crystalline film 160 is shown as flowing from a topmost layer of the nucleus 125. Because of a reduced curvature shape resulting from multiple layers of the nucleus 125 at a curvature from a layer coupled to a surface of the substrate 110 that enters the apertures 150, at some point, the nucleus 125 can be a single layer, the crystalline film 160, that is able to support its own weight due to its crystalline structure and extend across a cross sectional area of the nanoapertures 150, Thus, as should be understood, controlling the temperature of the first gas 135 and the second gas 140 continues the reaction at least until a formation of a crystalline film 160 of a solid product of the gases extends from the nucleus 125 and fills a cross-sectional area of at least a subset of the nanoapertures 150.

In some embodiments, controlling the temperature is performed as a function of a diameter of the nanoaperture. In some embodiments, controlling the temperature is performed as a function of a ratio between the diameter of the nanoaperture and a layer of thickness of the crystalline film. In some embodiments, controlling the temperature is performed as a function of a geometry of the cross-sectional area of the nanoaperture, the geometry selected from a group consisting of circular, ovular, rectangular, polygonal, curvilinear, or a combination thereof.

In some embodiments, and as illustrated in FIG. 1B, the first gas 135 includes a sulfur vapor, and the second gas 140 includes a molybdenum dioxide vapor. In such embodiments, the nucleus includes molybdenum disulfide. In some embodiments, the nucleus is coupled to the substrate via a membrane 113.

In some embodiments, and as illustrated in FIG. 1B, the substrate is a target substrate 114, and the method of manufacturing may further include positioning a backing substrate 116 in parallel arrangement with the target substrate 114, the arrangement of the target substrate 114 and the backing substrate 116 defining a gap 175 in which the first gas 135 flows at a controllable rate into at least a subset of the craters of the array of crater structures, wherein the gap 175 has a dimension that controls a flow rate of the first gas 135 into at least the subset of craters sufficient for the first gas 135 to react with the second gas 140 to produce the crystalline film 160 at at least the subset of the nanoapertures 150.

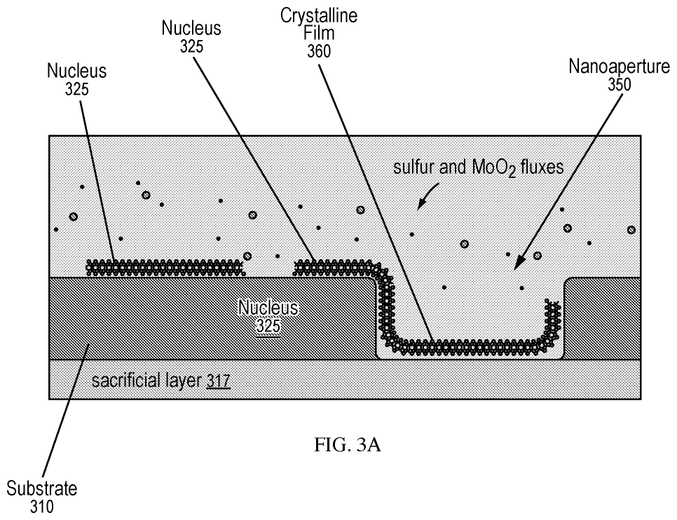

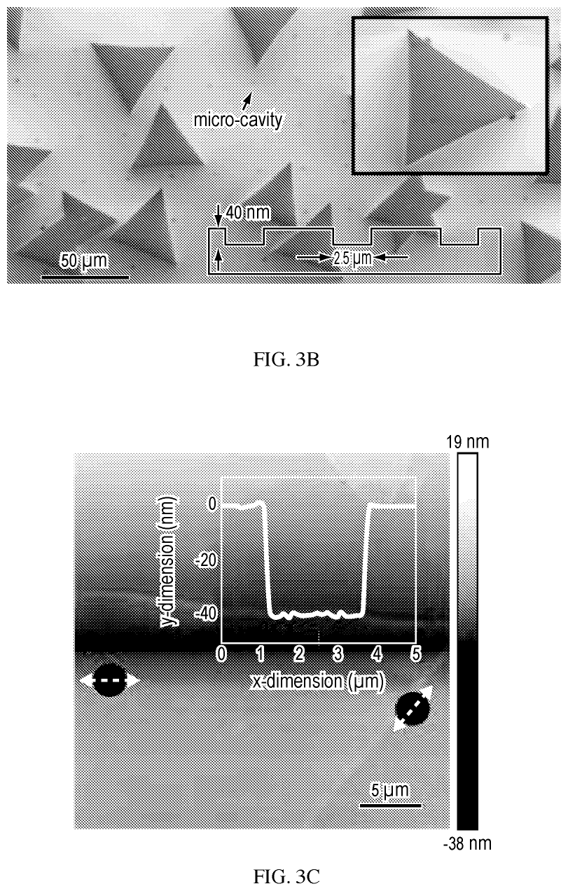

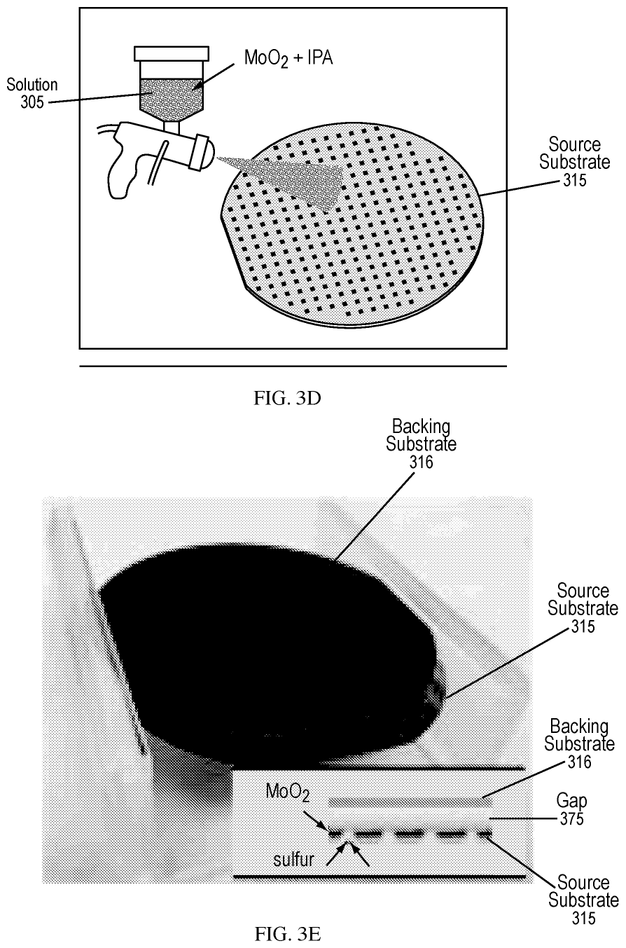

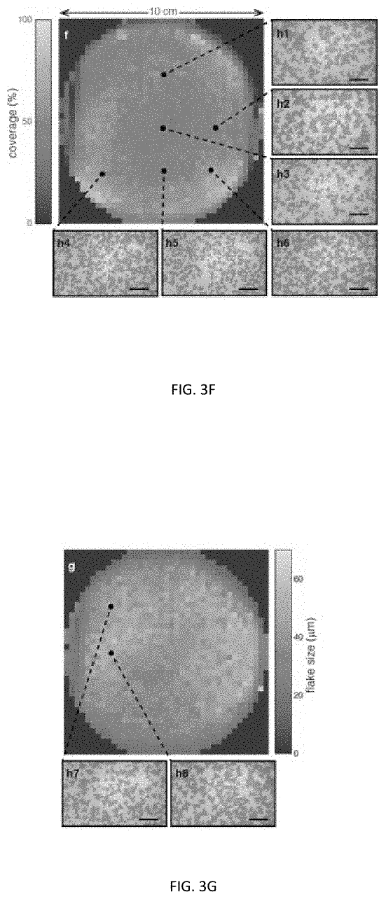

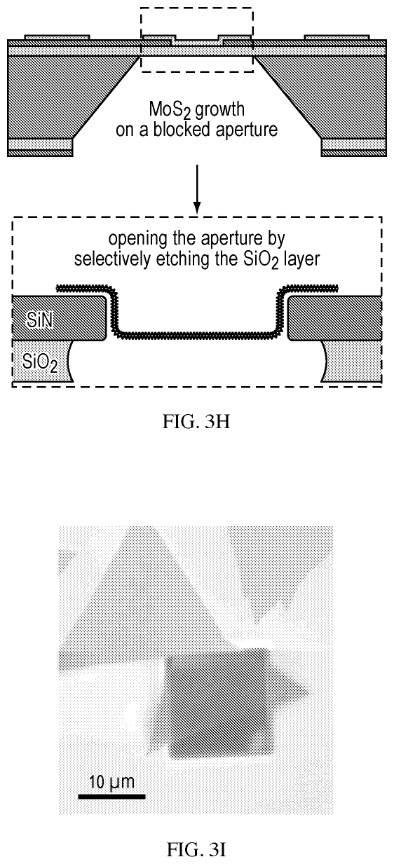

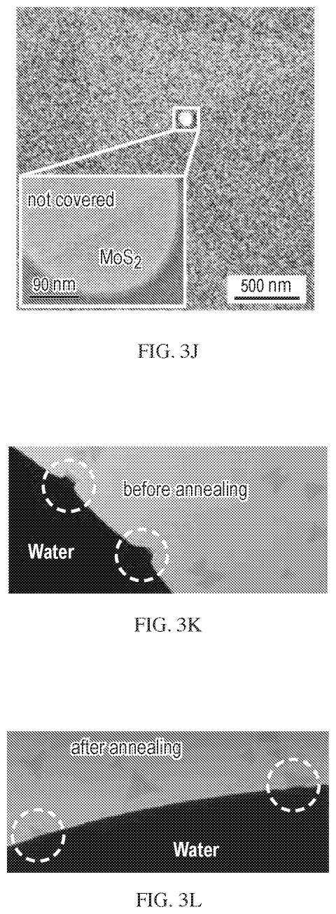

In some embodiments, and as illustrated in FIG. 1B and FIG. 3D, the substrate is a backing substrate 116, and the method of manufacturing further comprises positioning a source substrate 115, 315 in parallel arrangement with the backing substrate 116. The arrangement of the backing substrate 116 and the source substrate 115, 315 define a gap 175 in which the first gas flows at a controllable rate. The gap 175 is configured to retain the first gas 135 for a time sufficient for the first gas 135 to react with the second gas 140 to produce the crystalline film 160 at at least the subset of the nanoapertures 150. The embodiments may further include a source substrate 115, 315, and the method of manufacturing may further comprise (i) coating the source substrate 115, 315 with a chemical agent 305 that produces the second gas 140 at a given temperature and (ii) aligning the source substrate 115, 315 in offset parallel arrangement of the target substrate 114 in a plane opposite the target substrate 114 relative to a plane of the backing substrate 116.

Embodiments may further include removing a sacrificial layer 317, illustrated in FIG. 3A, that is coupled to the substrate 310 at a location between a given crater structure and a corresponding crystalline film 360. As will be illustrated in FIG. 3H, the sacrificial layer 317 will be removed, thereby leaving the crystalline film, in which a nanoaperture (not shown) can be created to enable molecules to flow therethrough.

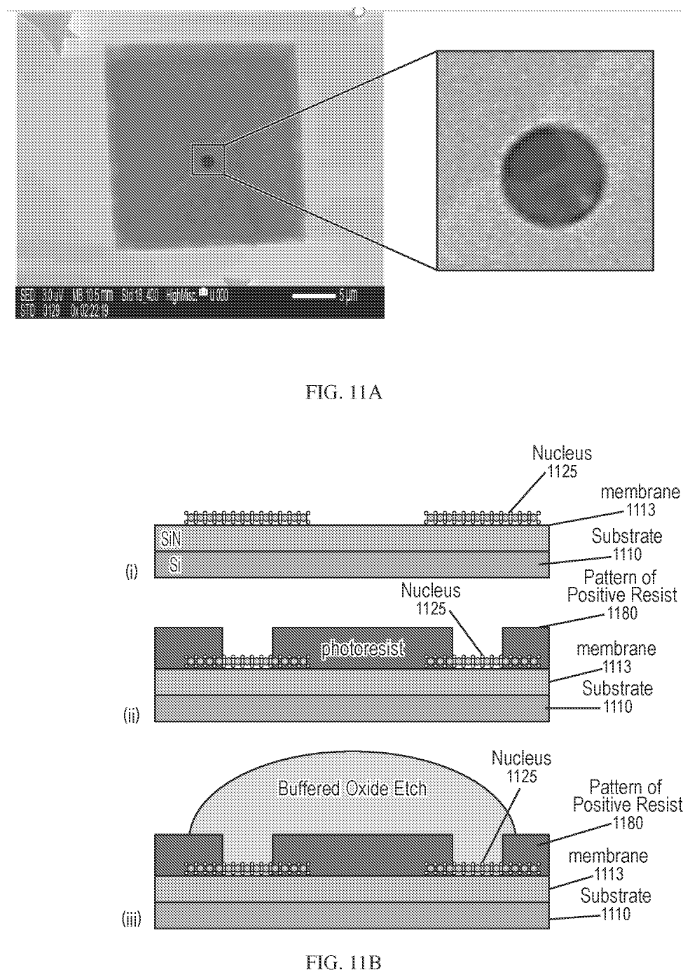

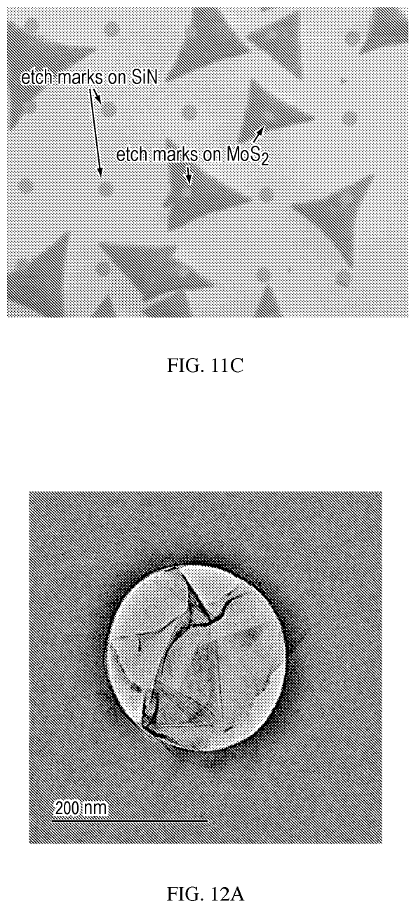

Some embodiments, as seen in FIG. 11B, further comprise pre-forming the substrate 1110 by pre-applying a pattern of positive resist 1180 on the substrate 1110 and exposing the substrate 1110 to an electron beam to form the array of the crater structures in the substrate 1110. Some embodiments further comprise exposing the substrate 1110 to a solution containing between about 5% to about 10% hydrogen for up to ten hours at pressure ranging from 50 Torr to 100 Torr at a temperature sufficient to stabilize crystals 1125 in the cross-sectional area of the nanoapertures.

In some embodiments, and as illustrated in FIG. 1B and FIG. 1E, exposing the substrate 110 to the first gas 135 and the second gas 140 is performed for a length of time known to induce controlled growth of the nucleus 125 at the nanoapertures 150, enabling the crystalline film 160 to fill a cross-sectional area of the nanoapertures 150. Some embodiments, as illustrated in FIG. 1B and FIG. 1E, further comprise applying an electric field to the crystalline film 160 at a level that produces a nanopore 155 therethrough.

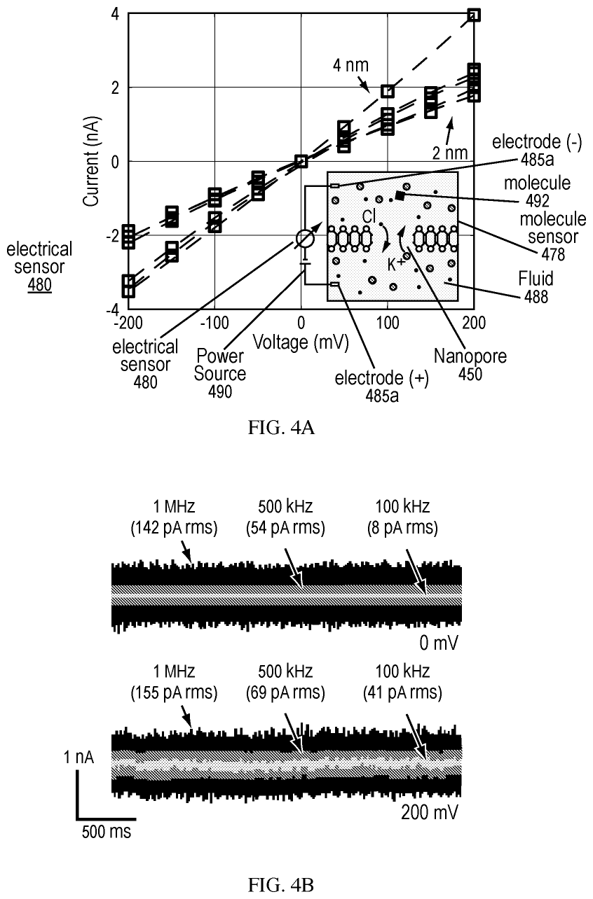

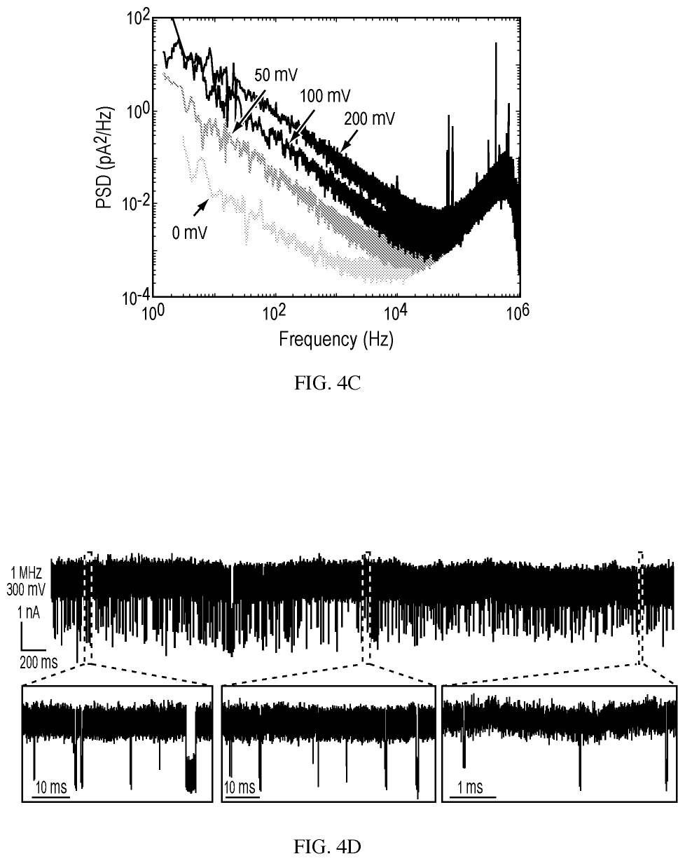

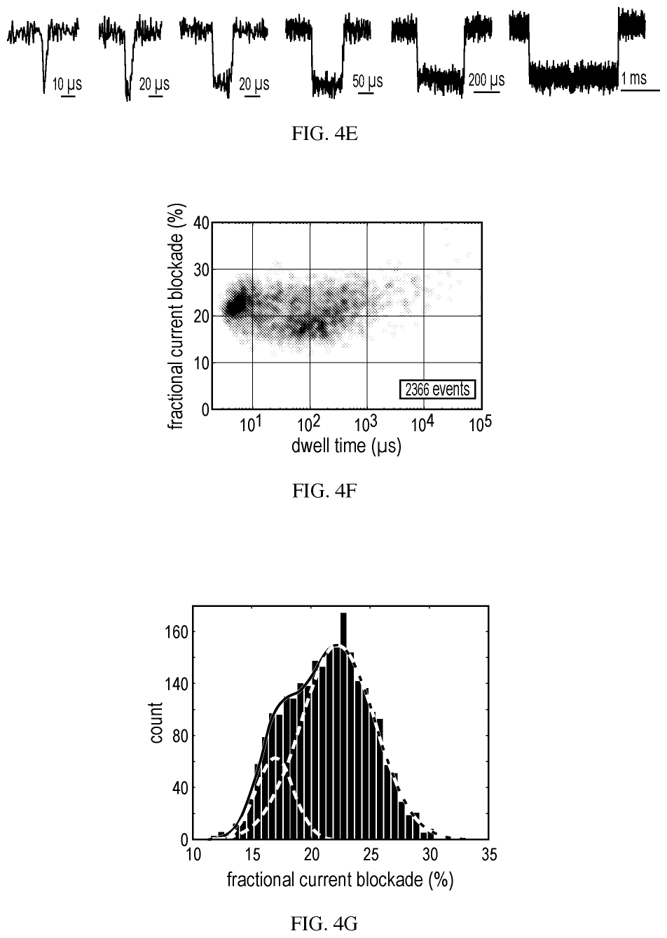

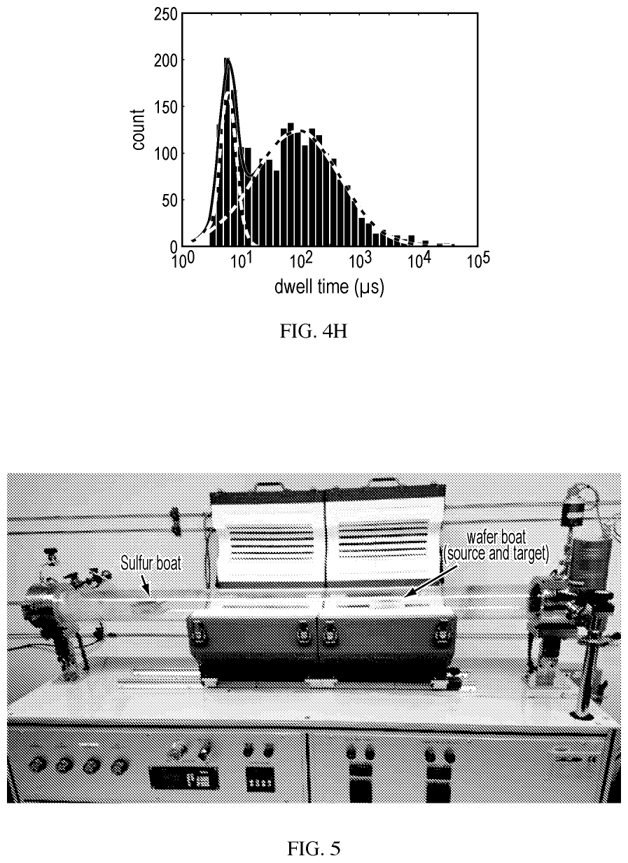

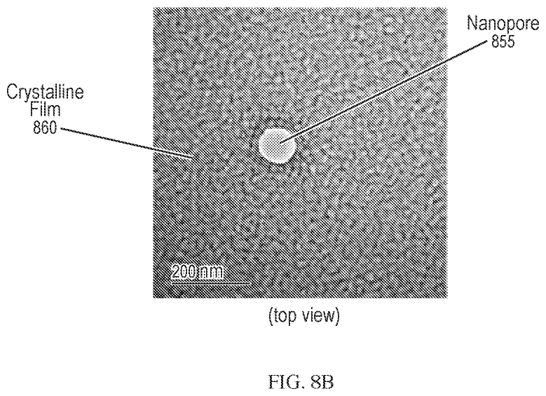

Some embodiments, as illustrated in FIG. 1A, further comprise separating the array of crater structures 145 into individual components 120, wherein each component 120 includes a respective portion of the substrate 110, a respective crater 145, and crystalline film. The component of FIG. 8A is a component that is provided by separating it from a larger substrate of components, as described in reference to FIG. 1A. Some embodiments of the method of manufacturing further comprise packaging an individual component 120 into a housing (not shown) that forms a molecule sensor, described below in reference to FIG. 4A.

In an embodiment, and as illustrated in FIG. 8A-B, a component for a molecule sensor comprises a substrate 810, a nucleus 825, and a crystalline film 860. The substrate 810 defines a crater structure (not shown) and nanoaperture 850 through the substrate 810 aligned with the crater structure (i.e., enabling a fluid to flow into and out of the crater structure via the substrate 810). The nucleus 825 is coupled to the substrate 810 at the nanoaperture 850 and forms a curvature 861 into the nanoaperture 850. The crystalline film 860 extends from the nucleus 825 and fills a cross-sectional area of the nanoaperture 850.

In some embodiments, and as illustrated in FIGS. 8A-B, the crystalline film defines 860 a respective nanopore 855 through which a molecule may pass. In some embodiments, as seen in FIGS. 8A-B, the nanopore 855 has a diameter from about 50 nm to about 200 nm. In some embodiments, the curvature 861 is defined by layers of the nucleus 825 at the nanoaperture 850. The nucleus 825 may be a product of a sulfur vapor and a molybdenum dioxide vapor. The nucleus may be coupled to the substrate 810 via a membrane.

FIG. 4A is a diagram of another example embodiment of the invention in the form of a molecule sensor 478 that includes a substrate, a nucleus, and a crystalline film as described herein, such as in reference to FIGS. 8A-B. In the example embodiment of FIG. 4A, the substrate defines a crater structure and nanoaperture aligned therewith. The nucleus is coupled to the substrate and forms a curvature into the nanoaperture. The crystalline film extends from the nucleus and fills a cross-sectional area of the nanoaperture. The crystalline film also defines a nanopore with a dimension sufficient to enable a molecule to pass therethrough. The crystalline film may be at least partially below a surface of the substrate within the nanoaperture.

The molecule sensor 478 includes electrodes 485a, 485b that, when energized by a power source 490, cause ions (e.g., and Cl⁻ and K⁺) to produce a current flow within a fluid 488 in the molecule sensor. In turn, a molecule 492, such as a DNA molecule that is negatively charged, is drawn toward the positive electrode 485a. As the molecule 492 enters, is within, or passes out of the nanopore 455, an electrical sensor 480 that is configured to detect a change of an electrical signal that indicates that the molecule entered, is within, or passed through the nanopore 455, senses such an event. It should be understood that the molecule sensor 478 may include simple processing and report a sensed event (e.g., a molecule exiting the nanopore 455). The molecule sensor 478 may also perform complex processing and perform signal processing that can identify molecule(s) based on an electronic signature as a function of an electrical signal waveform (not shown) associated with the electrodes 485a, 485b or otherwise associated with the nanopore 455.

As demonstrated in example embodiments, MoS₂ can directly grow in free-space without a substrate, to completely cover and seal 0.5-2 μm nanoapertures. Nonetheless, understanding the growth mechanism remains elusive, and lack of sufficient control over microscopic quantities in terms of substrate quality and growth conditions has so far resulted in typically thick nanoaperture coverage with many number of layers at low yields. As a result, given the small number of chips that can be accommodated in a 1″ CVD tube that was used for MoS₂ growth, no more than a few devices per run was achieved. Example embodiments expand this concept and demonstrate scaled-up and transfer-free synthesis of high quality freestanding MoS₂ membranes on nanoapertures. The method allows fabrication of many membrane devices (˜200 devices from a 4″ substrate) in a single growth run with the possibility of loading multiple substrates at the same time, which combined with voltage-assisted nanopore fabrication enables extensive MoS₂ nanopore studies. The first-principles quantum simulations elucidate the mechanism of freestanding membrane growth and provide fundamental insight into the role of nanoaperture size in successful formation of the freestanding membranes, which is linked to the observation of ring-like MoS₂ structures lining around the nanoaperture interior during growth.

As shown in example embodiments, leakage-free, robustly anchored 2D membranes on nanoapertures which contributes to high quality nanopore signals, also allowing investigations of 2D nanofluidic systems that advance the understanding of various anomalous transport behaviors at the nanoscale and the development of efficient filtration membranes. Example embodiments show a quantitative comparison of this method with another method which does not rely on vapor flow through the nanoapertures and guarantees uninterrupted growth of single MoS₂ crystals that span the nanoapertures, albeit at reduced yields. The fabrication yield of this non-selective growth method depends ultimately on surface coverage of the MoS₂ flakes, and thus achieving high-coverage growth on the entire 4″ substrate area is key to high-yield fabrication. Finally, example embodiments demonstrate the first MHz-bandwidth recordings of DNA translocation through MoS₂ nanopores, determining the fundamental impact of access resistance on the signal obtained during ultrafast DNA interaction with the pore (1-10 μs timescales). The methods presented herein allow high-yield MoS₂ device fabrication for high-throughput nanofluidics and nanopore studies.

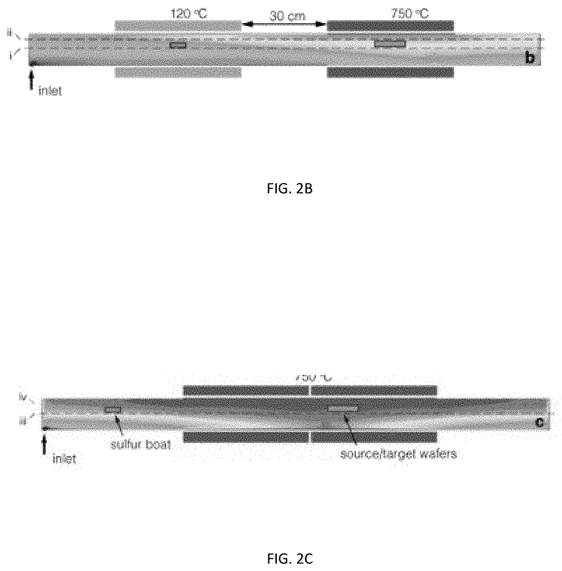

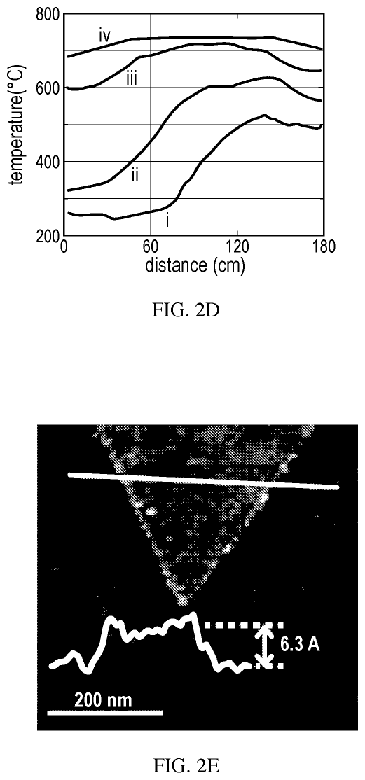

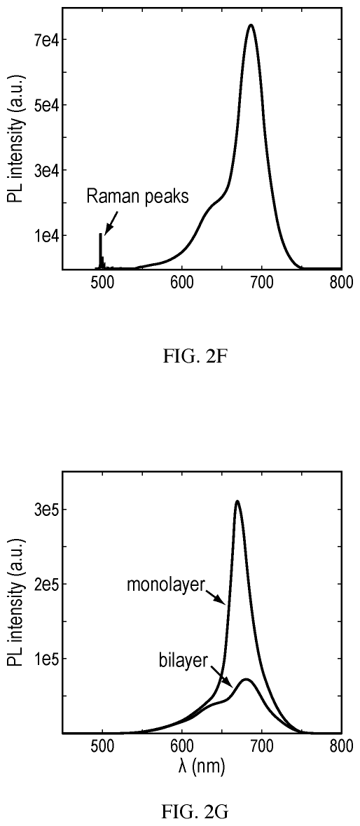

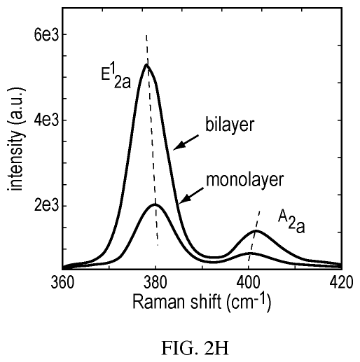

Scaled-up synthesis of molybdenum disulfide 2D crystals. There have been efforts for scaled-up growth of MoS₂ over large areas using CVD and metal-organic CVD (MOCVD) techniques. Generally, CVD growth results in higher crystal quality and a lower cost than other techniques such as molecular beam epitaxy or atomic layer deposition. Nonetheless, use of solid precursors for CVD growth poses numerous challenges that include vaporization timing and flux supply, which limit the spatial uniformity and batch-to-batch repeatability. This problem becomes even more critical when growth over a large area is intended, since this requires a uniform thermal and reagent flux zone across a large diameter tube. Two-furnace 5″ diameter CVD tube with a 4.5″ inner diameter is used, as depicted in FIG. 5, and the sulfur boat 217 and substrates 214, 215 are arranged in the tube 201 as shown in FIG. 2A. Sulfur powder is placed in a sulfur boat 217, and MoO2 powder is spread on a source substrate 215 and placed directly under the target substrate 214 (i.e., the substrate on which growth occurs) to provide a uniform MoO₂ supply during the growth phase.

Failure to grow in this large tube with furnaces arranged is shown in FIGS. 2A-H. FIGS. 2B-C show the difficulty of achieving the required MoS₂ growth temperature. The computational analysis of the tube, as seen in FIG. 2B, (see Methods section below) indicates that setting the left furnace to 120° C. (to melt the sulfur) and the right furnace to 750° C. (for MoO₂ sublimation and MoS₂ growth) with a gap in between results in strong density-gradient-driven secondary flows in the tube, which significantly contribute to heat loss from the tube. As a result, the intended temperature for growth at the backing substrate position cannot be achieved. Setting the temperature of both furnaces to 750° C. and placing them, as shown in FIG. 2C, supplies more heat to the growth region of the tube, reduces temperature loss near the backing substrate, and allows temperature to reach the intended value at the backing substrate site, while allowing sulfur sublimation outside of the left tube. This configuration is also associated with a reduced axial flow velocity in the tube, and a smoother stream. Unlike the case in small tubes (1- or 2-inch diameter), where laminar axial flows develop, in the case of 5″ diameter tubes large vortices can develop that fundamentally alter the temperature and flow profiles in the tube.

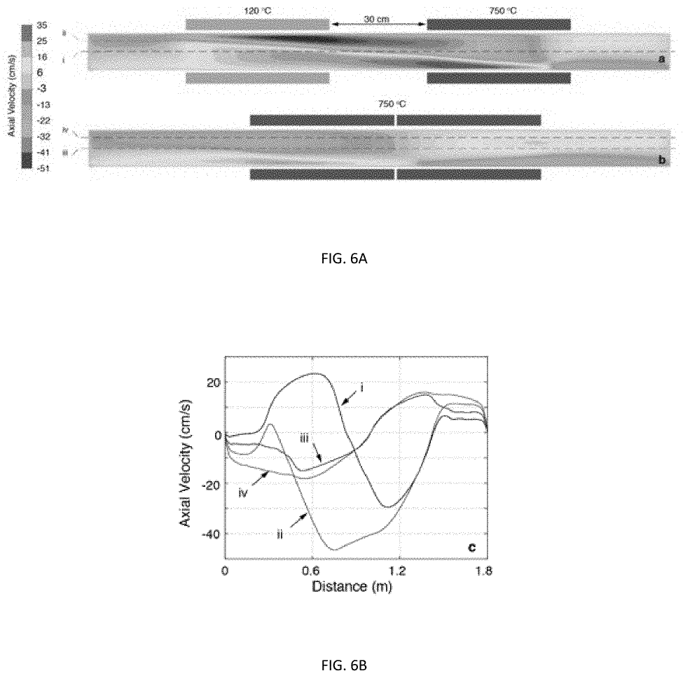

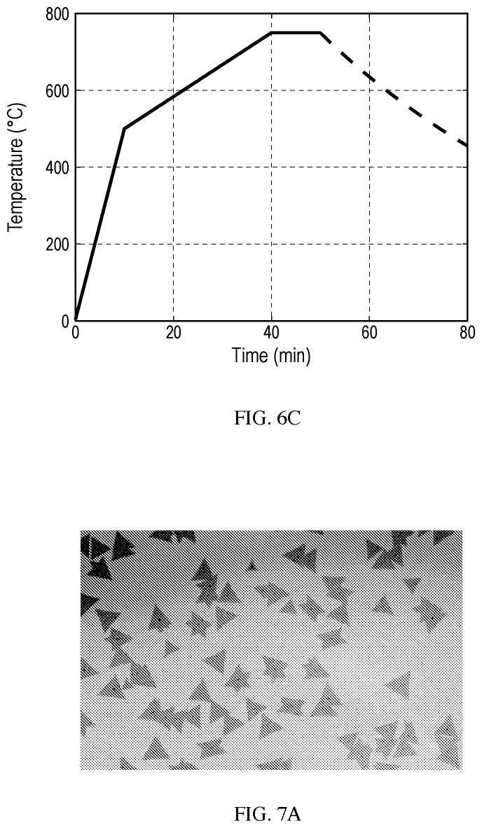

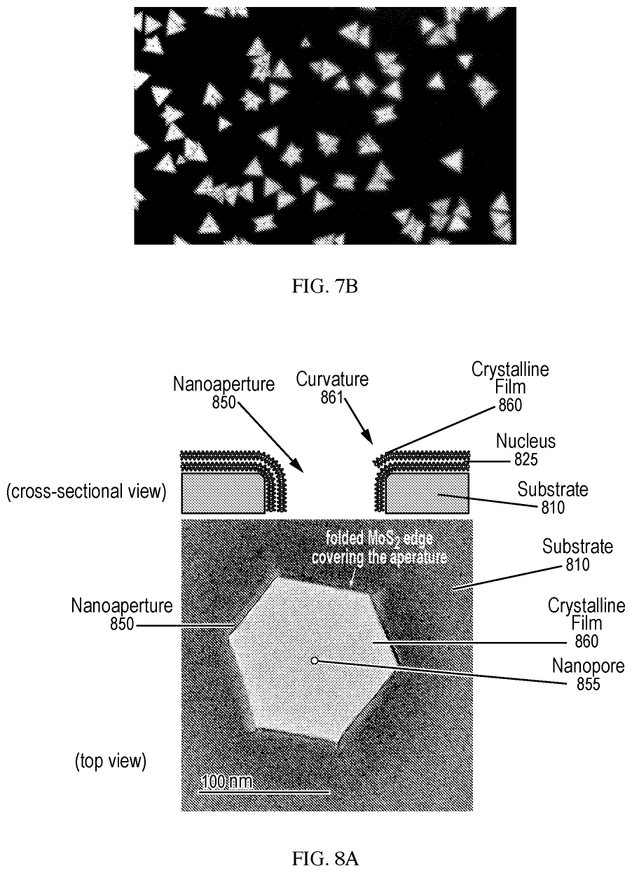

Upon addressing the heat loss from the tube, the temperature profile shown in FIG. 6C was used in a typical growth (see Methods section below). An example of MoS₂ flakes grown on a Si/SiO₂ substrate is shown in FIGS. 7A-B. Since the AFM and photoluminescence measurements are time consuming and can be done on a limited number of spots on the substrate, 2D maps of PL intensities can be used to examine very large areas over a short period of time. The microscope is equipped with a 470 nm collimated LED illumination (350 mW) and a 532 nm long pass emission filter to measure photoluminescence intensity from the entire microscope's field of view using a sensitive monochromatic CMOS camera (FLIR Grasshopper3). A PL image is shown in FIG. 7C wherein bright flakes are easily identifiable by their emission over a dark background, which is the characteristic of monolayer MoS₂. Darker areas observed at the center of some of the flakes indicate thicker MoS₂ layers in self-seeded crystals. Examination of the flakes by atomic force microscopy (Dimension FastScan, Bruker) in several regions on the substrate indicates a 0.6-0.8 nm flake thickness, as shown in FIG. 2E, which confirms monolayer growth. PL emission at the flake edges is brighter than the center of the flakes. A similar effect has been reported in WS₂ 2D crystals which was explained on the basis of chemisorption of oxygen atoms at the crystal edges. This effect is growth dependent, and in other cases the PL at the edge is darker than the center, as shown in FIGS. 7A-B. Further quantification of the flakes with PL spectroscopy, shown in FIG. 2F (λₑₓ=488 nm=2.54 eV), reveals a strong emission peak centered at 686 nm (1.81 eV). Such a monolayer growth can be guaranteed by carefully controlling the amount of MoO₂ as well as the gap between source and backing substrates. A similar growth quality was observed on SiN substrates as evidenced by high PL intensities, as shown in FIG. 2G. Photoluminescence intensity significantly decreases for a bilayer flake. The Raman shift for a monolayer MoS₂ on the SiN substrate (λₑₓ=488 nm) was measured to be 20.1 cm⁻¹, as shown in FIG. 2G.

Selective growth on nanoapertures. After achieving uniform growth across the entire substrate area, next is to achieve substrate-scale growth of freestanding MoS₂ membranes across nanoapertures. Understanding the growth of 2D materials on non-planar surfaces is essential in developing methods for direct formation of freestanding 2D membranes. When out-of-plane substrate features (such as grooves, or holes) are smaller than typical flake size, the crystal growth responds to their presence through different scenarios. MoS₂ is grown on substrates that have been processed to contain arrays of 5×5 mm chips that have freestanding SiN membranes with a 50-100 nm diameter circular nanoaperture present on each membrane, as depicted in FIGS. 1A and 1B. The nanoapertures 150 provide a unique opportunity for selective growth—by locally mixing a first gas 135, such as a sulfur vapor, and a second gas 140, such as MoO₂ vapor, from either side of the membranes 113, MoS₂ preferentially grows on or near the nanoapertures, as shown in FIG. 1B. However, controlling the vapor flux at the nanoaperture is a very critical factor in formation of the freestanding membranes. The optimal MoO₂ vapor concentration at the nanoapertures was achieved by setting the gap between the source and backing substrates to 5 mm, shown in FIG. 1B. In order to control the sulfur flux, the nanopore substrate is backed with a backing substrate 116 (also referred to as a wafer) so that the two substrates 114, 116 are in close contact and the only gap 175 between them is due to flatness of the substrates 114, 116. Based on the prime-grade substrates' total thickness variation (TTV), the average gap between the two substrates is estimated to be δ≈10 μm. The two substrates were pressed against each other using a ceramic plate and a set of ceramic nuts/bolts as shown in FIG. 1C. The sulfur flux can be further adjusted by choice of the sulfur boat's 217 surface area, as well as its location along the tube 201 which dictates its temperature, as illustrated in FIG. 2A.

Inspection of the membranes under an optical microscope after growth indicates growth of large MoS₂ flakes on all SiN membranes, which confirms the selective growth mechanism, as shown in FIG. 1D. Nonetheless, large-flake growth does not guarantee the formation of freestanding MoS₂ membranes across the nanoapertures. Further inspection of the nanoapertures with a transmission electron microscope (TEM) indicates a peculiar growth within the nanoaperture that results in formation of freestanding membranes, as seen in FIG. 1D. The formation of multiple layers of MoS₂, which comprise the crystalline film 160 on the substrate 110, is observed, resembling a multi-shell MoS₂ tube that decorates the nanoaperture interior. This observation is structurally very similar to earlier reported seeded growth where growth starts from a fullerene-like multi-shell structure formed around the seeds. This side-wall MoS₂ growth is clearly identifiable under TEM, as shown in FIG. 1D, and is the signature of the selective growth mechanism where the two reagents only meet at the nanoaperture. In order to conform to the circular shape of the nanoaperture, several fault lines appear around the nanoaperture. In a rare case in which growth around the nanoaperture was terminated only after formation of the first layer, shown in FIG. 1E, a faceted edge was created which further verified the folding of flakes, also referred to herein as a curvature 161, at the nanoaperture 150 edge, which is also shown in FIG. 8A. Formation of such MoS₂ tubes is an indication of geometry-constrained growth which can be further employed for developing other 2D nanofluidic systems. The structure of flakes grown on sidewalls is schematically shown in FIG. 1F, in which the MoS₂ layers within the nanoaperture 150 bend at the edge and extend on the SiN membrane surface. In fact, the TEM images of the nanoaperture vicinity always shows a number of layers decorated in various forms around the nanoapertures 150, as shown in FIG. 1G. From purely thermodynamical arguments, this folding can only occur if the van der Waals energy between layers exceeds the energy penalty of bending, otherwise flakes do not bend and grow across the nanoapertures. FIG. 1H displays the nanoaperture curvature 161where the freestanding membrane and the side-wall layers meet, and shows how a completely sealed crystalline film 160 is formed. Finally, the high angle annular dark field (HAADF) imaging of a membrane containing a nanopore, seen in FIG. 1I, enables precise identification of the pore shape and lattice structure. Here, the Mo atoms are evident in this image as bright white dots. The expected hexagonal lattice of the monolayer MoS₂ membrane was confirmed by the electron diffraction pattern (inset).

Smoothness of the nanoaperture edge and its very close vicinity area is critical for formation of the freestanding membranes. During the microfabrication process, this area may be roughened which results in excessive nucleation during the MoS₂ growth. Protecting the membranes with polymethyl methacrylate (PMMA) before etching the underlying oxide layer, combined with brief reactive ion etching of the SiN membranes after SiO₂ was etched during substrate preparation significantly improves likelihood of formation of MoS₂ membranes (see Methods section below). Conversely, treating the SiN membrane with buffered oxide etch or phosphoric acid prevents growth of flakes and consistently results in growth of a mesh-like structure at the surface, shown in FIG. 8B. The average fabrication yield of freestanding MoS₂ membranes in three consecutive full-substrate growths was found to be 75%. To obtain this number, one breaks away pieces from three substrates produced in different runs and inspected 142 chips under TEM, 107 of which contained MoS₂ freestanding membranes completely spanning the nanoapertures, 28 chips were not fully covered, and 7 chips showed excessive growth at the nanoaperture. This substrate-scale fabrication yield is similar to the recently reported transfer-based method of fabrication of MoS₂ membranes on 3″ substrates. Yet, the method allows simultaneous MoS₂ growth on multiple 4″ nanopore substrates in a single run, which significantly improves the throughput. In order to estimate the number of layers in the freestanding membranes, nanopores are drilled in the MoS₂ membranes using TEM to be able to probe the pore edge structure. Membranes are typically 1-3 layer thick, and many of them contain monolayer regions of arbitrary sizes. Such membranes are ideal for nanopore sensing purposes, as while the pore can be created in a thinner region, the overall electric noise is reduced due to the presence of thicker regions on the freestanding portions of the membranes. The observation of the presence of 1-to-3-layer thick membranes is consistent with the notion that MoS₂ membrane formation is self-limiting, i.e., reagent flux through the nanoaperture is blocked once a sealed membrane forms.

Electron microscopy images shown in FIGS. 1D and 1G-H allow for the development of a qualitative mechanism of the steps of the CVD process that eventually lead to the development of a single-layer membrane (referred to herein as the crystalline film 160) at the top. Due to the specific experimental setup, MoS₂ initially grows from both sides of a membrane 113, such as SiN, on the target substrate 114. This growth leads to the development of multiple MoS₂ layers espousing the contours of the membrane 113 (e.g., SiN) on the target substrate 114 until a crystalline film 160 (e.g., MoS₂) grows across the nanoaperture 150. This step leads to drastic reduction in CVD activity since the membrane effectively stops feedstock from reaching the top of the layer from below. Thermodynamic arguments were used to gain further insight into the reasons why there is a crossover from the growth of bent MoS₂ layers into that of a quasi-flat one.

Nonselective growth of membranes. Selective MoS₂ membrane growth on nanoapertures is an effective method of scaled-up synthesis, which requires delicate preparation of the nanoaperture, as well as control of vapor flux through the nanoapertures. On the other hand, the fact that a growing MoS₂ flake follows the surface morphology, inspired an alternative sacrificial-layer based fabrication scheme, shown in FIG. 4A. The SEM and AFM images of MoS₂ grown on a model substrate entirely patterned with micro-cavities (2.5 μm diameter, 40 nm deep, and 25 μm pitch) confirm that when MoS₂ flakes meet a groove, growth is not interrupted, instead following the surface profile and conformally coating the substrate, shown in FIGS. 3B-C. Therefore, by growing MoS₂ on a microcavity nanowell backed by a sacrificial layer and establishing an orthogonal chemistry for selectively etching the sacrificial layer without etching the MoS₂, a freestanding membrane was formed. In this method, formation of the membranes relies on coincidental growth of flakes on cavities, and is referred to as the nonselective growth. In this case, the likelihood of freestanding membrane formation is proportional to the overall coverage of the substrate with MoS₂ flakes, and it becomes essential to achieve high coverage growths.

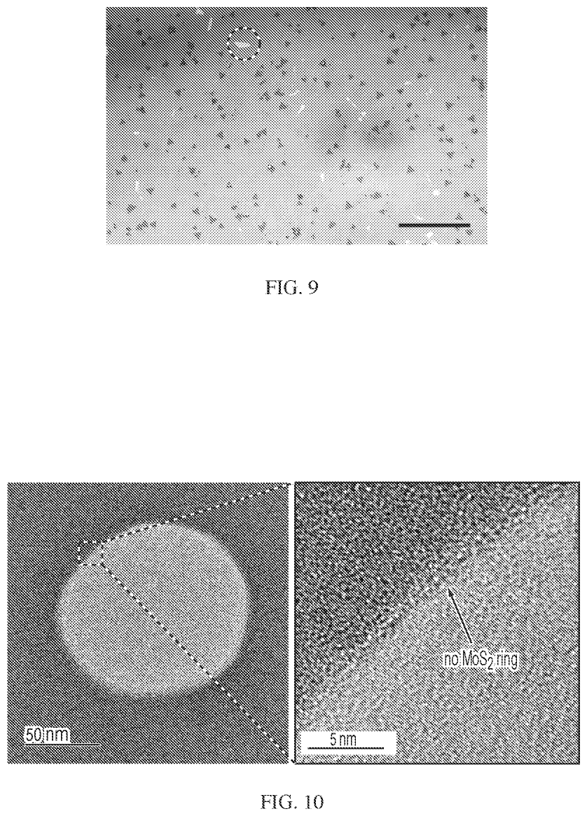

Supplying uniform flux of both reagents onto the backing substrate is key to obtaining a high coverage and uniform growth, or else a patchy growth is resulted. By spraying a MoO₂/IPA suspension mix solution 305 on a perforated substrate, as shown in FIG. 3D, not only was MoO₂ dispensed evenly on the entire source substrate 315, but also the holes on this source substrate 315 allowed sulfur transport through the substrate, seen in FIG. 3E, and uniform showering on the backing substrate 316 (see Methods section below for details). FIGS. 3F-G show the coverage map and the crystal size map of a typical growth with this method, along with microscope images of the flakes on different spots on the substrate. Here, 50% of the substrate surface area is covered by MoS₂ flakes without using any seeding, with some regions reaching 100% film coverage, shown in FIG. 9. The triangular shapes of all the flakes indicate very uniform supply of MoO₂ and sulfur vapors over the entire area with the method, or else hexagonal- and star-shaped flakes would be observed. Example embodiments demonstrate similar growth for Si/SiO₂ substrates, Si/SiN substrates, and Si/SiO₂/SiN substrates which are the substrates used for SiN nanoaperture fabrication. Flake size and coverage can be conveniently regulated by controlling the MoO₂ mass and the gap between the source and backing substrates.

In fabrication of the nanopore substrates, the SiN layer is always deposited on a SiO₂ layer, which serves to reduce the capacitive noise of the devices (see Methods section below). This SiO₂ layer can also serve as the sacrificial layer 317, owing to the orthogonal chemistry that it forms with MoS₂ and the buffered oxide etch (BOE), which enables selective etching of the SiO₂ layer and release of MoS₂ freestanding membranes, shown in FIGS. 3A and 3H. Therefore, in preparation of the substrates, after etching the nanoapertures through the SiN layer, this SiO₂ layer is not removed until after the MoS₂ grown on the substrate, as depicted in FIGS. 3H-I. This method results in very uniform coating of the nanoapertures, without excessive nucleation and growth around the nanoaperture, as depicted in FIG. 3J and FIG. 10. The nanoapertures blocked by silicon oxide are invisible to the growth, as neither side-wall growth on the nanoaperture (the MoS₂ tubes) occurs in this method, nor the direction of growth changes when a flake meets the nanoaperture edge, seen in FIG. 3J.

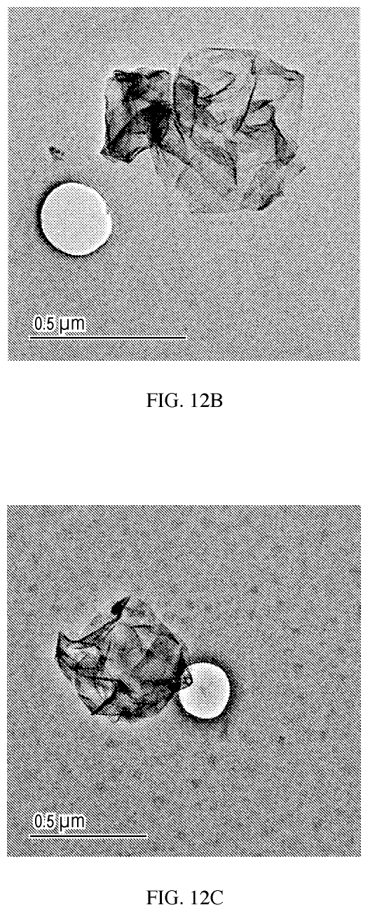

Upon exposure to aqueous medium, buffers, or organic solvents flakes were observed to wrinkle, roll up, or in some cases float. Annealing substrates in argon environment (containing 5% hydrogen) at 400° C. at 50 Torr for 5 hours was observed to resolve this problem. This is an essential step in the process and stabilizes the MoS₂ flakes in solution. This step reduced the relative hydrophilicity of the flakes as evidenced by water contact angle experiments. FIG. 3I shows a water droplet placed on a substrate before annealing. In some example embodiments, water at the droplet edge is observed while it spreads on the flakes indicating smaller contact angles on the flakes compared with the substrate. After droplet edge recedes due to evaporation, the flakes are observed to have rolled up. After annealing, however, the water contact angle on the substrate and flakes became very close, as shown in FIGS. 3K-L, and the flakes remained adherent to the substrate. The UV ozone treatment helps to maintain flake adhesion to the substrate during immersion in solution, which suggests that matching surface energies between the flakes and the substrates promotes their stability upon immersion, while mismatched surface energies promote flake roll-up and wrinkling upon immersion. Despite the simplicity of this method and the smooth nanoaperture coverage achieved, there is some variability in terms of etch resistance of the MoS₂ to BOE, which ultimately reduces the yield of this method, shown in FIGS. 11A-C. Addressing this seemingly growth dependent etch resistance is beyond the scope of this paper. Finally, after removing the SiO₂ layer and deionized water rinsing, if the nanoapertures are too large (200 nm) the membrane may be pulled through the nanoapertures, as shown in FIGS. 12A-C, and therefore smaller nanoaperture sizes are required to achieve stable freestanding MoS₂ using this method.

Nanopore sensing. Next, ion current leakage and electrical noise through freestanding MoS₂ membranes by performing trans-membrane conductance measurements (see Methods) is characterized. After mounting the devices in a fluidic flow-cell a freestanding MoS₂ membrane separates two electrolyte-filled reservoirs, such that in the presence of a nanopore, application of an electric field across the membranes creates a steady ionic current, shown in FIG. 4A. In the absence of a nanopore, any DC current recorded can be attributed to the membrane leakage. Nonetheless, measuring the current leakage is difficult, and, in accordance with example embodiments, the MoS₂ membrane is fully wet on both sides of the membrane. This is particularly challenging in 2D nanopores due to their hydrophobicity. For MoS₂ membranes a rapid pretreatment of the membrane with acetonitrile (CH₃CN) can significantly facilitate subsequent hydration. Membrane wetting can be confirmed in the absence of a nanopore by observing a longer RC time constant in the transient capacitive current when a voltage step is applied, than for a membrane occluded with an air bubble. In the absence of a nanopore no appreciable conductance can be measured for a wet membrane (<10 pS, which has contributions from the capacitive response too), indicating a complete membrane seal in the nanoapertures. Another indication of wetting is the fact that a nanopore could be punctured by a brief application of a <1V voltage bias, presumably through a dielectric breakdown (also referred to electrochemical reaction) mechanism. In some example embodiments, voltage-induced poration is used to form nanopores since voltage induces the highest electric field on the thinnest regions of the membranes, thereby favoring the formation of single-layer-thick pores.

The i-v curves of seven different nanopores in the diameter range of 2-4 nm are shown in FIG. 4A. Three on these pores were drilled using TEM and others were formed by dielectric breakdown. Nanopore conductance is used to estimate the pore diameter through

$$G = {σ ( \frac{1}{D} + \frac{4 l}{π {D}^{2}} )}^{- 1} ,$$

with σ being the electrolyte conductivity, D the pore diameter, and l the pore thickness. For these ultrathin MoS₂ membranes access resistance is the dominating term and thus the conductance can be estimated by G=σD. Ionic current traces recorded at 0 mV and 200 mV and lowpass-filtered at various cutoff frequencies, along with corresponding rms noise values are shown in FIG. 4B. Below 100 kHz, the rms noise significantly increases with increasing the applied voltage, while above 100 kHz rms noise values are similar.

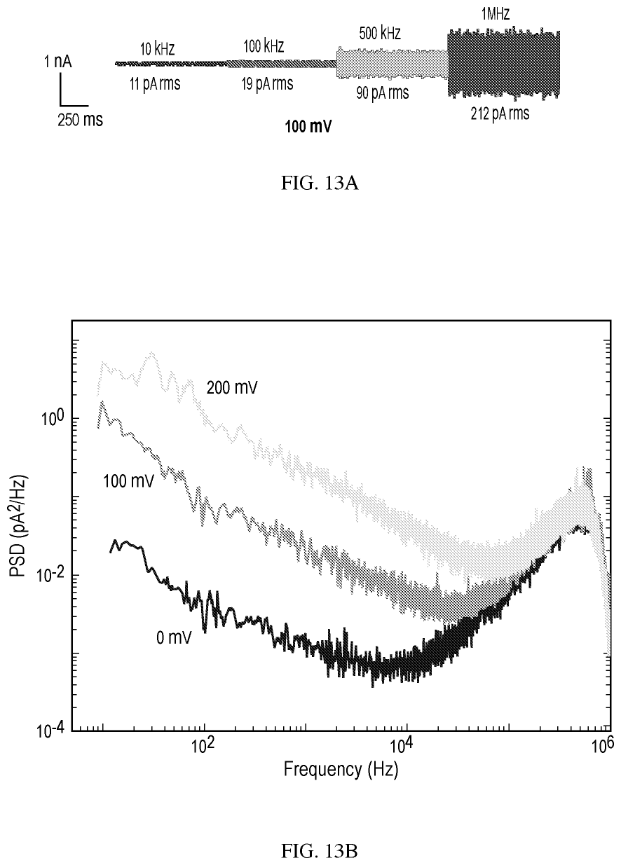

This can be further observed in the noise spectra at different voltages, which indicate larger contributions to the overall noise from the low-frequency regime, particularly as voltage increases, as shown in FIG. 4C. This 1/f noise is a characteristic of 2D nanopores which has been previously reported for graphene and MoS₂ nanopores. Recent studies suggest that the 1/f noise originates from the surface conduction of the nanopore, and adsorption-desorption of ions at the pore surface which is coupled with the long-lasting excursions of the ions in the reservoirs. There are variations in the contribution of the low frequency components to the total rms noise from device to device, as shown in FIG. 13A-B, possibly due to the area of the monolayer region.

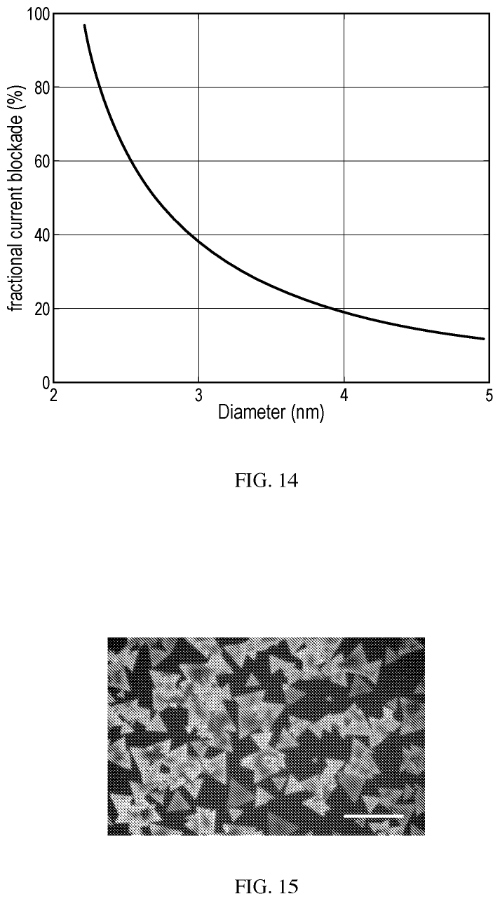

FIG. 4D shows a current trace of translocation of 500 bp dsDNA through MoS₂ nanopores at 300 mV, with selected magnified views shown below the trace. The pore diameter which was formed with dielectric breakdown was estimated to be 4 nm. The 1 MHz amplifier bandwidth (Chimera Instruments LLC, see Methods section below) enabled recording very fast translocations down to ˜2 μs. Several representative translocation events with dwell times in the μs to ms range are shown in FIG. 4E. The scatter plot of fractional current blockades versus dwell times including 2,366 events is shown in FIG. 4F. The current trace used in the scatter plot was lowpass filtered at 500 kHz to allow better fitting of the events to square pulses. Two distinct populations are evident in the scatterplot. The fast population to DNA collision with the pore and the slow population is attributed to translocation events. The likelihood of collision with pore as opposed to translocation is influenced by the pore shape, which is not known. The fractional current blockade of the translocating events is 17%, as shown in FIG. 4G, which agrees well with the theoretical estimation seen in FIG. 14. In order to estimate the blockade, one assumes the access resistance of a nanopore (R_{acc}=1/σD) during translocation is modified as R_{acc}=1/σD_{eff} with D_{eff}=\sqrt{D²−d_{dsDNA}²} (d_{dsDNA}=2.2 nm is the cross-sectional diameter of dsDNA).

The collision events are blocking slightly higher currents (22%). The mean dwell times for the two populations are 6_{-2.3}^{3.8} μs and 88₋₈₀⁸³⁷ μs.

In summary, example embodiments demonstrate MoS₂ growth over substratescale areas as large as 4″ with high coverage without any need for seeding, and presented two methods for scaled-up fabrication of MoS₂ freestanding membranes. The study encompasses over 450 CVD runs, commencing with optimization of uniform growth on different substrate substrates without nanoapertures (Si/SiO₂, Si/SiN, and Si/SiO/SiN), and then proceeding with selective growth optimization on 34 substrates containing SiN nanoapertures and 10 substrates with SiN micro-apertures, as shown in FIG. 11A. Over 1,050 chips were individually inspected using TEM, the most reliable method to confirm freestanding MoS₂ membrane formation. Selective growth proceeds by nucleation near or at the nanoapertures, and results in the formation of multiple flakes growing around the nanoaperture, followed by freestanding membranes that span the nanoaperture once the radius of curvature is too large to bend around the nanoaperture curvature. Focusing the vapor-phase reagents to the nanoaperture is critical here to the selective growth, which enables formation of freestanding membranes with 75% yields across a 4″ substrate. Outlined in some example embodiments are the roles of smoothness of the nanoaperture vicinity, controlled reagent flux, and criticalnanoaperture size on the successful formation of freestanding membranes. Further, introduced in an alternative method is the elimination of the trans-pore vapor flux suppresses excessive growth around the nanoaperture, resulting in large MoS₂ flakes. These MoS₂ membranes exhibit excellent seals, preventing ion flow through nanopore-free membranes and very low noise as compared with other pores in transferred 2D membranes. Demonstrated in some example embodiments is the first high-bandwidth (1 MHz) measurements of DNA transport through a MoS₂ nanopore, highlighting the contribution of access resistance on the overall signal in sub-nm nanopore thicknesses. The scaled-up method for synthesis of MoS₂ membranes accelerates various single-molecule measurements with 2D nanopores, and advances 2D nanofluidics research such as development of 2D filtration membranes.

### Methods

Substrate-scale fabrication of freestanding SiN membranes with nanopores. The substrates were made by deposition of 50 nm thick medium-stress SiN on 300-μm-thick and 500-μm-thick Si (100) substrates that contain a 2-μm-thick wet thermal SiO₂ grown on them. Deposition of silicon nitride was performed at Lurie Nanofabrication Facility (LNF). Some example embodiments employ the use of e-beam lithography with positive resists (ZEP 520A, ZEON Corporation, Tokyo, Japan) to pattern the entire substrates with circles with diameters in the range of 50-100 nm, 5 mm pitch. After etching the SiN with RIE, photolithography and backside alignment is used to pattern the other side of substrate to expose windows for potassium hydroxide (KOH) etching. After an RIE step to etch the SiN, a single-side etcher is used to remove the 2-μm-thick SiO₂ layer using buffered oxide etch (BOE 6:1, J.T. Baker Chemicals, #5569-03) for 40 minutes. Next, the substrates were etched by KOH (30% w/w, Fisher Chemical, #P246-3) at 70° C. to obtain the freestanding SiN/SiO₂ membranes. In order to remove the SiO₂ layer under the SiN membranes, the membrane side of the substrate was spin-coated with PMMA (495 PMMA A4, MicroChem) and baked on a hotplate at 160° C. for 2 minutes to protect the nanoaperture vicinity against BOE, and then the underlying silicon oxide layer was etched by BOE. The PMMA was later removed by warm acetone immersion (60 minutes, 45° C.). Fabrication of substrates with micro-apertures is very similar, the only difference being the use of photolithography to pattern the micro-apertures. After fabrication substrates were cleaned using a hot piranha solution for 15 minutes (H₂SO_{4:}H₂O₂, 2:1), thoroughly rinsed with deionized water, and baked on a hotplate at 200° C. Substrates were briefly etched by RIE (Technics Micro-RIE, series 800) for 10 seconds using Ar/SF₆ gas mixture (50 W, 200 mTorr) before growth.

Chemical Vapor Deposition. MoO₂ (Molybdenum(IV) oxide, 99%, Sigma-Aldrich, #234761) and sulfur powders (Alfa Aesar, −100 mesh, 99.5%, #33394) were used in CVD growths after carefully weighing. 40 mg MoO₂ is used in a typical growth. Given the small amount of powder that must be spread over a large area (40 mg MoO₂), the MoO₂ powder is mixed with isopropanol (IPA, Fisher Chemical, #A416-4) and sprayed over the source substrate which yielded excellent uniformity, as shown in FIG. 3D. Fast evaporation of IPA after spray is the key reason behind this choice of solvent. The amount of MoO₂ was verified by weighing the source substrate before and after the spray, following substrate drying. Alternatively, the MoO₂ can be manually spread on the source substrate in a uniformly thin layer. Uniform supply of sulfur is an even more important factor for large scale uniform MoS₂ growth, as sulfur vapor can only diffuse from the substrate edges toward the center which creates a radial non-uniformity with higher coverage at the edges and lower coverage and sulfur deficient growth towards the center. A source substrate 315 is fabricated containing an array of tapered square through-holes that allowed diffusion of sulfur through the source substrate 315, as shown in FIGS. 3D-E. These holes were 1.5 mm wide on the large end of the taper, 0.8 mm wide on its small end, with 5 mm pitch. This technique significantly improved the MoS₂ coverage and flake size uniformity across substrates.

Growth was carried out in Argon environment in a 5-inch OD CVD tube 201 (PlanarGROW-5M, PlanarTech; see, e.g., FIG. 5 and FIG. 2A), with 750 Torr pressure, and 1000 SCCM flow rate, with a temperature profile shown in FIG. 6C. The optimal gap between the source and backing substrates for high-coverage growth was found to be 5 mm. A change in the gap size as large as 0.5 mm, and in the amount of MoO₂ as much as 5 mg results in completely distinguishable growths, in terms of MoS₂ flake size and coverage. Increasing the furnace temperature to up to 800° C. further improved the coverage due to increasing sublimation rates of MoO₂. Since both furnaces have to operate at 750° C., one relies on the radiative and convective heat from the furnaces to melt the sulfur. A quartz sulfur boat was placed at a 23cm distance from the edge of the left furnace, as shown in FIG. 5. Given the strong temperature gradient in the CVD tube outside the furnace zone, location of the boat is critical in determining the sulfur kick-in time, which is essential for a good growth. The boat is placed at a height in the tube above the axis where convection becomes the dominant mode of heating. By placing the boat on-axis and closer to the edge of furnace, radiation effects become dominant, which can overheat the sulfur boat.

Following a CVD run substrates cannot be reused, since complete removal of the Mo-containing particles from the substrates after the first growth was not possible, which resulted in these particles seeding growth in the next run, which biased the growth evaluation. Moreover, when a growing flake meets these particles, the growth is terminated, and as a result smaller flakes are observed when reusing a substrate even after extensive cleaning. Statistics of flake size and the substrate coverage with flakes were obtained by programming a microscope equipped with a motorized stage. The entire area of each substrate was scanned at a 2.5 mm pitch and at each location an image was captured. These images were fed to an image processing program written in MATLAB which recognized flakes in an unsupervised manner. The results were then assembled to show the growth map on each substrate and quantify the growth.

While example embodiments have been particularly shown and described, it will be understood by those skilled in the art that various changes in form and details may be made therein without departing from the scope of the embodiments encompassed by the appended claims.

What is claimed is:

1. A method of manufacturing a component for a molecule sensor, the method comprising:
exposing a substrate to a first gas and a second gas, the substrate defining an array of crater structures and nanoapertures aligned therewith, the gases being at a temperature that induces a reaction that produces a nucleus coupled to a surface of the substrate at the nanoapertures and forms a curvature into the nanoapertures; and
controlling the temperature of the first gas and the second gas to continue the reaction at least until a formation of crystalline film of a solid product of the gases, extending from the nucleus, fills a cross-sectional area of at least a subset of the nanoapertures.

2. The method of manufacturing of claim 1, wherein controlling the temperature is performed as a function of a diameter of the nanoaperture.

3. The method of manufacturing of claim 1, wherein controlling the temperature is performed as a function of a ratio between the diameter of the nanoapertures and a layer of thickness of the crystalline film.

4. The method of manufacturing of claim 1, wherein controlling the temperature is performed as a function of a geometry of the cross-sectional area of the nanoaperture, the geometry selected from a group consisting of circular, ovular, rectangular, polygonal, curvilinear, or a combination thereof.

5. The method of manufacturing of claim 1, further comprising flowing the first gas with an inert gas across a first surface of the substrate and evenly distributing the second gas across the second surface of the substrate and within the crater structures.

6. The method of manufacturing of claim 1, wherein the first gas includes a sulfur vapor and the second gas includes a molybdenum dioxide vapor, and wherein the nucleus includes molybdenum disulfide

7. The method of manufacturing of claim 1, wherein the nucleus is coupled to the substrate via a membrane.

8. The method of manufacturing of claim 1, wherein the substrate is a target substrate, and further comprising positioning a backing substrate in parallel arrangement with the target substrate, the arrangement of the target substrate and the backing substrate defining a gap in which the first gas flows at a controllable rate into at least a subset of the craters of the array of crater structures, wherein the gap has a dimension that controls a flow rate of the first gas into at least the subset of craters sufficient for the first gas to react with the second gas to produce the crystalline film at at least the subset of the nanoapertures.

9. The method of manufacturing of claim 6, further comprising a source substrate, and wherein the method of manufacturing further comprises (i) coating the source substrate with a chemical agent that produces the second gas at a given temperature and (ii) aligning the source substrate in offset parallel arrangement of the target substrate in a plane opposite the target substrate relative to a plane of the backing substrate.

10. The method of manufacturing of claim 1, further comprising removing a sacrificial layer that is coupled to the substrate at a location between a given crater structure and a corresponding crystalline film.

11. The method of manufacturing of claim 1, further comprising pre-forming the substrate by pre-applying a pattern of positive resist on the substrate and exposing the substrate to an electron beam to form the array of crater structures in the substrate.

12. The method of manufacturing of claim 1, further comprising exposing the substrate to a solution containing between about 5% to about 10% hydrogen for up to ten hours at pressure ranging from 50 Torr to 100 Torr at a temperature sufficient to stabilize crystals in the cross-sectional area of the nanoapertures.

13. The method of manufacturing of claim 1, wherein exposing the substrate to the first gas and the second gas is performed for a length of time known to induce controlled growth of the crystalline film at the nanoapertures.

14. The method of manufacturing of claim 1, further comprising applying an electric field to the crystalline film at a level that produces a nanopore therethrough.

15. The method of manufacturing of claim 1, further comprising separating the array of crater structures into individual components that includes a respective portion of the substrate, a respective crater, and crystalline film.

16. The method of manufacturing of claim 14, further comprising packaging an individual component into a housing that forms a molecule sensor.

17. A component for a molecule sensor, the component comprising:
a substrate defining an array of crater structures and nanoapertures aligned therewith;
a nucleus coupled to the substrate at the nanoapertures and forms a curvature into the nanoapertures; and
a crystalline film extending from the nucleus and filling a cross-sectional area of at least a subset of the nanoapertures.

18. The component of claim 16, wherein the crystalline film defines a respective nanopore through which a molecule may pass.

19. The component of claim 16, wherein the nanopore has a diameter from about 50 nm to about 200 nm.

20. The component of claim 16, wherein the curvature is defined by layers of the nucleus at the nanoaperture.

21. The component of claim 17, wherein the nucleus is a product of a reaction between a sulfur vapor and a molybdenum dioxide vapor.

22. The component of claim 17, wherein the nucleus is coupled to the substrate via a membrane.

23. A molecule sensor, comprising:
a substrate defining a crater structure and nanoaperture aligned therewith;
a nucleus coupled to the substrate that forms a curvature into the nanoaperture; and
a crystalline film that extends from the nucleus and fills a cross-sectional area of the nanoaperture, the crystalline film defining a nanopore with a dimension sufficient to enable a molecule to pass therethrough.

24. The molecule sensor of claim 23, wherein the crystalline film is at least partially below a surface of the substrate within the nanoaperture.

25. The molecule sensor of claim 23, further comprising:
electrodes that, when energized, cause the molecule to pass through the nanopore; and
a sensor configured to detect a change of an electrical signal, the change of the electrical signal indicating that the molecule entered, is within, or passed through the nanopore.
